(19) United States
(12) Patent Application Publication
(10) Pub. No.: US 2003/0119066 A1
(43) Pub. Date: Jun. 26, 2003

(54) Diagnostic kit for detecting immunogenic response and method of screening

(75) Inventors: Erwin Ludo Roggen, Lyngby (DK); Nina Teeres Nilsson, Kavlinge (SE); Steffen Ernst, Bronshoj (DK); Shamkant Anant Patkar, Lyngby (DK); Esben Peter Friis, (US)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 10/264,559

(22) Filed: Oct. 4, 2002

Related U.S. Application Data
(60) Provisional application No. 60/330,289, filed on Oct. 18, 2001.

(30) Foreign Application Priority Data
Oct. 5, 2001 (DK) 2001 01473

(51) Int. Cl. A61K 39/00; G01N 33/53; G01N 33/537; G01N 33/543

(52) U.S. Cl. 435/7.1; 435/7.93; 424/185.1

(57) ABSTRACT

The present invention relates to a kit for predicting binding of specific antibodies to potential immunogens. The kit comprises antigenic peptide sequences having less than 26 amino acids, said antigenic peptide sequences being capable of binding antibodies specific for structural epitopes contained on potential immunogens. The antigenic peptide sequences are immobilized on a solid support.

### CROSS-REFERENCE TO RELATED APPLICATIONS

This application claims under 35 U.S.C. 119 priority from Danish application no. PA 2001 01473 filed Oct. 5, 2001 and the benefit of U.S. provisional application No. 60/330,289 filed Oct. 18, 2001, the contents of which are fully incorporated herein by reference.

### BACKGROUND OF THE INVENTION

1. Field of the Invention

The present invention relates to a kit for predicting binding of a specific antibody to at least one potential immunogen, as well as a high throughput screening method for testing the presence of antibodies specific for at least one structural epitope comprised in at least one potential immunogen. Further the invention relates to a use of the kit and/or the high throughput screening method for predicting binding of specific antibodies, in one or more samples, to at least one or more potential immunogen(s). Still further the invention relates to a vaccine comprising an antigenic peptide sequence corresponding to a structural epitope comprised in a potential immunogen. Finally the invention relates to a use of at least one antigenic peptide sequence corresponding to a specific structural epitope in at least one potential immunogen for the preparation of a vaccine, a method for preparing such a vaccine and use of such a vaccine.

2. Description of Related Art

An increasing number of proteins, including enzymes, are being produced industrially, for use in various industries, housekeeping and medicine. Being proteins they are likely to stimulate an immunological response in man and animals, including an allergic response.

As the food market becomes more globalized, the average consumer runs a higher risk of encountering unexpected allergens. These foreign allergens add up to the increased use of mixtures of proteins as well as additives by a more and more industrialized food production.

Humans or animals may become sensitised to allergens e.g. by inhalation, direct contact with skin and eyes, or injection. The general mechanism behind an imunnogenic, and in particular an allergic response, is divided in a sensitisation phase and a symptomatic phase. The sensitisation phase involves a first exposure of a human or animal to an allergen. This event activates specific T- and B-lymphocytes, and leads to the production of allergen specific IgE antibodies (in the present context the antibodies are denoted as usual, i.e. immunoglobulin E is IgE etc.). These IgE antibodies eventually facilitate allergen capturing and presentation to T-lymphocytes at the onset of the symptomatic phase. This phase is initiated by a second exposure to the same or a resembling antigen. The specific IgE antibodies bind to the specific IgE receptors on mast cells and basophils, among others, and capture at the same time the allergen. The polyclonal nature of this process results in bridging and clustering of the IgE receptors, and subsequently in the activation of mast cells and basophils. This activation triggers the release of various chemical mediators involved in early as well as late phase reactions of the symptomatic phase of allergy.

For certain forms of IgE-mediated allergies, a therapy exists, which comprises repeated administration of allergen preparations called ‘allergen vaccines’ (Int. Arch. Allergy Immunol., 1999, vol. 119, pp1-5). This leads to reduction of the allergic symptoms, possibly due to a redirection of the immune response away from the allergic (Th2) pathway and towards the immunoprotective (Th1) pathway (Int. Arch. Allergy Immunol., 1999, vol. 119, pp1-5). However, for most of the allergies avoiding contact with the allergen still is the only available treatment.

Whatever therapeutic strategy, a proper diagnosis of the allergy, i.e. proper identification of the challenging allergen, is required to optimise either the ‘allergen vaccination’ therapy or the ‘abstinence’ approach.

The diagnosis of humans or animals with allergic symptoms is not well developed. Moreover, there is a gap between the identification of single IgE-binding allergens and the quantitative risk assessement.

Numerous tests exist for determination of the biological potency of molecules or mixtures. Challenge of human patients are considered as closest to the relevant biological response, i.e. elicitation of an actual immunogenic, and in particular an allergic response, albeit under controlled and safe circumstances. Skin tests obviously involves the skin mast cells, which must be sensitised by IgE in order to respond to the offending allergen. A biological in vitro system is the sensitised basophil granulocyte. This system mimicks the sensitized mast cell present in the relevant target organ of the patient. Moving even further away from the actual patient, basophils from, e.g. cord blod, of a non-allergic donor, may be used as a reagent. These cells must be sensitised by IgE derived from the actual patient.

Presently, double blind placebo controlled food challenge (DBPCFC) is considered valid in diagnosing food allergy, and compared to this gold-standard, there are many examples of in vivo and in vitro diagnostic tools which produce misleading results. The reason for the low specificity of these tests is the extensive cross-reactions between species, and between environmental allergens and food allergens.

A pure system can be obtained by immunochemical assays detecting IgE-allergen binding, directly or indirectly, by inhibition designs. These assays should preferentially include single allergen specific IgE epitopes in order to allow direct risk assessment.

Several similar techniques for localization of B-cell epitopes are disclosed by Walshet et al, J. Immunol. Methods, Vol. 121, 1275-280 (1989), and by Schoofs et al., J. Immunol., Vol. 140, 611-616 (1987). All of these documents relate to identification of linear epitopes.

Slootstra et al., Molecular Diversity, Vol. 2, pp. 156-164 (1996) discloses the screening of a semi-random library of synthetic peptides for their binding properties to three monoclonal antibodies.

WO 99/47680 (ALK-ABELLÓ) discloses the identification and modification of B-cell epitopes by protein engineering.

WO 00/26230 (Novozymes A/S) describes the use of phage-display libraries for identifying linear as well as conformational epitope sequences and patterns on proteins. This information is stored in a database, and provides a rational approach for identifying antigenic and allergenic areas on proteins.

Conformational/structural epitopes are less likely to be present on different immunogens and the use of such epitopes in diagnosis or characterization og immunoglubulins from a human or animal will therefore give a more precise answer without the problems of cross reactivity.

Identification of such conformational/structural epitopes can be used in the context of the present invention in order to precisely identify interactions between such conformational epitopes and specific antibodies and provides a fast method of screening a large number of different allergenic epitopes at the same time.

### SUMMARY OF THE INVENTION

The present invention relates to a kit for predicting binding of a specific antibody to at least one potential immunogen, comprising

a) at least one antigenic peptide sequence comprising less than 26 amino acids wherein said antigenic peptide sequence corresponds to a structural epitope comprised in the at least one potential immunogen and the antigenic peptide sequence is capable of binding at least one antibody specific for the structural epitope comprised in the said potential immunogen, and

b) solid support suitable for immobilizing the at least one antigenic peptide sequence.

A second aspect of the present invention relates to a high throughput screening method for testing the presence of antibodies specific for a structural epitope comprised in at least one potential immunogen of interest, comprising

a) providing one or more antigenic peptide sequences comprising less than 26 amino acids wherein said one or more antigenic peptide sequence(s) correspond(s) to one or more structural epitopes comprised in the at least one potential immunogen and the antigenic peptide sequence(s) is/are capable of binding at least one antibody specific for the structural epitope(s) comprised in the said potential immunogen(s),

b) immobilizing the one or more antigenic peptide sequences to a suitable solid support,

c) adding the specific antibodies from a sample, and

d) detecting binding of specific antibodies to any of the one or more antigenic peptide sequences.

A third aspect of the present invention relates to a use of the kit for predicting binding of a specific antibody in a sample to at least one potential immunogen, wherein binding of antibody to at least one antigenic peptide sequence corresponding to at least one structural epitope on the at least one potential immunogen is tested.

A fourth aspect of the present invention relates to a use of the high throughput screening method for screening antibodies from at least one sample.

A fifth aspect of the invention relates to a vaccine comprising at least one antigenic peptide corresponding to a structural epitope comprised in at least one potential immunogen and said antigenic peptide sequence being capable of binding at least one antibody specific for the structural epitope comprised in the potential immunogen.

A sixth aspect of the invention relates to a method of preparing a vaccine comprising adding to a liquid medium at least one antigenic peptide sequence, corresponding to a structural epitope comprised in at least one potential immunogen and said antigenic peptide sequence being capable of binding at least one antibody specific for the structural epitope comprised in the potential immunogen.

A seventh aspect of the invention relates to a use of at least one antigenic peptide sequence, corresponding to a structural epitope comprised in at least one potential immunogen and said antigenic peptide sequence being capable of binding at least one antibody specific for the structural epitope comprised in the potential immunogen, for the preparation of a vaccine.

An eighth aspect of the invention relate to a use of the vaccine of the invention for the treatment of a human or an animal.

### BRIEF DESCRIPTION OF DRAWINGS

FIG. 1 shows the antibody binding capacity of selected peptides. The antibody binding capacity of the two linear peptide sequences, RRFANDHTR (light gray bars) and RRFSNATRA (dark gray bars), were tested in an ELISA assay, by measuring optical density, OD. The sequences were tested for binding to antibodies in sera raised against different proteins. The different proteins are marked by capital letters A through H. A=Alcalase®, B=Savinase®, C=Subtilisin Novo®), D=Carezyme®) (cellulase), E=Laccase, F=Natalase® (amylase), G=SP722 (amylase), H=Lipolase® (lipase).

### DEFINITIONS

Prior to a discussion of the detailed embodiments of the invention, a definition of specific terms related to the main aspects of the invention is provided.

The term “epitope” is defined as an antigenic determinant and is a set of amino acids on a protein that are involved in an immunological response, such as antibody binding or T-cell activation. It is the simplest form or smallest structural area on a complex antigen molecule that can combine with an antibody or T lymphocyte receptor. An epitope must be at least 1 kD (about 10 amino acids) in order to be immunogenic. Epitopes can be linear or conformational/structural.

The term “linear epitope” is defined as an epitope composed of amino acid residues that are contiguous on the linear sequence of amino acids (primary structure).

The term “epitope sequence” is defined as the amino acid residues which makes up the epitope.

The term “conformational or structural epitope” is defined as an epitope composed of amino acid residues that are not all contiguous. The epitope is thus composed of separated parts of one or more linear sequences of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’.

The term “antibody binding peptide” is defined as a peptide that binds with sufficiently high affinity to antibodies. In particular the antibody binding peptide is linear, but it may also be circular. Identification of ‘antibody binding peptides’ and their sequences constitute the first step of the method of this invention.

The term “epitope pattern” means a consensus sequence of antibody binding peptides. An example is the epitope pattern A R R>R. The sign “>” or “<” in this notation indicates that the aligned antibody binding peptides may or may not include one or more non-consensus amino acids between the second and the third arginine.

The term “anchor amino acids” means the individual amino acids of an epitope pattern.

The term “immunogen” is a substance that is able to induce a humoral antibody and/or cell-mediated immune response rather than immunological tolerance. The term ‘immunogen’ is sometimes used interchangeably with ‘antigen’, yet the term specifies the ability to stimulate an immune response as well as to react with the products of it, e.g. antibody. By contrast, ‘antigen’ is reserved by some to mean a substance that reacts with antibody. The principal immunogens are proteins and polysaccharides, free or attached to microorganisms.

The terms “immunogenic/immunogenicity” means the capacity to induce humoral antibody and/or cell-mediated immune responsiveness.

The term “donor protein” means the protein that was used to raise antibodies for identification of antibody binding sequences, hence the donor protein provides the information that leads to the epitope patterns. The donor protein may e.g. be the parent protein or a part of it.

The term “acceptor protein” is the protein, whose 3D-structure is used to fit the identified epitope patterns and/or to fit the antibody binding sequences. Hence, the acceptor protein may e.g. be the parent protein or a part of it.

“Monospecific polyclonal antibodies” are polyclonal antibodies that are specifically binding to a certain epitope, and hence are monospecific. The polyclonal nature of these antibodies is explained by the fact that a number of antibody-producing B-cell clones may produce antibodies to similar epitopes (but with the same epitope pattern as the epitope of interest), that bind to the epitope of interest, though with lower affinity.

The term “immunogenic response” including allergic response, used in connection with the present invention, is the response of an organism to a compound, which involves IgE mediated responses (Type I reaction according to Coombs & Gell). It is to be understood that sensibilization (i.e. development of compound-specific IgE antibodies) upon exposure to the compound is included in the definition of “immunogenic response”.

An “epitope area” is defined as the amino acids situated close to the epitope sequence amino acids. Particularly, the amino acids of an epitope area are located <5 Angstroms from the epitope sequence. Hence, an epitope area also includes the corresponding epitope sequence itself. Modifications of amino acids of the ‘epitope area’ can possibly affect the immunogenic function of the corresponding epitope.

“Environmental allergens” are protein allergens that are present naturally. They include pollen, dust mite allergens, pet allergens, food allergens, venoms, etc.

“Commercial allergens” are protein allergens that are being brought to the market commercially. They include enzymes, pharmaceutical proteins, antimicrobial peptides, as well as allergens of transgenic plants.

The term “specific polyclonal antibodies” means polyclonal antibodies isolated according to their specificity for a certain antigen, e.g. the protein backbone.

### DETAILED DESCRIPTION OF THE INVENTION

Identification of Antibody Binding Peptides and Epitope Pattern

A first step required to carry out the present invention is to identify peptide sequences, which bind specifically to antibodies.

Antibody binding peptide sequences can be found by testing a set of known peptide sequences for binding to antibodies raised against the donor protein. These sequences are typically selected, such that each represents a segment of the donor protein sequence (Mol. Immunol., Vol. 29, pp.1383-1389 (1992); Am. J. Resp. Cell. Mol. Biol., Vol. 22, pp. 344-351 (2000)). Also, randomized synthetic peptide libraries can be used to find antibody binding sequences (Slootstra et al., Molecular Diversity, Vol. 2, pp. 156-164 (1996)).

In a particular method, the identification of antibody binding sequences may be achieved by screening a display package library, particularly a phage display library. The principle behind phage display is that a heterologous DNA sequence can be inserted in the gene coding for a coat protein of the phage (WO 92/15679). The phage will make and display the hybrid protein on its surface where it can interact with specific target agents. Such target agent may be antigen-specific antibodies. It is therefore possible to select specific phages that display antibody-binding peptide sequences. The displayed peptides can be of predetermined lengths, for example 9 amino acids long, with randomized sequences, resulting in a random peptide display package library. Thus, by screening for antibody binding, one can isolate the peptide sequences that have sufficiently high affinity for the particular antibody used. The peptides of the hybrid proteins of the specific phages which bind protein-specific antibodies characterize epitopes that are recognized by the immune system.

The antibodies used for reacting with the display package are particularly IgE antibodies to ensure that the epitopes identified are IgE epitopes, i.e. epitopes inducing and binding IgE. In a particular embodiment the antibodies are polyclonal antibodies, optionally monospecific antibodies.

For the purpose of the present invention particularly polyclonal antibodies are used in order to obtain a broader knowledge about the epitopes of a protein.

It is of great importance that the amino acid sequence of the peptides presented by the display packages is long enough to represent a significant part of the epitope to be identified. In a particular embodiment of the invention the peptides of the peptide display package library are oligopeptides having from 5 to 25 amino acids, particularly at least 8-12 amino acids, such as 9 amino acids. For a given length of peptide sequences (n), the theoretical number of different possible sequences can be calculated as 20ⁿ. The diversity of the package library used must be large enough to provide a suitable representation of the theoretical number of different sequences. In a phage-display library, each phage has one specific sequence of a determined length. Hence an average phage display library can express 10⁸-10¹² different random sequences, and is therefore well-suited to represent the theoretical number of different sequences.

Hence, in one embodiment of the invention, antigenic peptides for use in the kit of the invention are obtained by screening a random peptide library with antibodies raised against any immunogen of interest and sequencing the amino acid sequence of antibody binding peptides or the DNA sequence encoding the peptides. Once such sequences have been established the peptides may be prepared/produced.

The antibody binding peptide sequences can be further analysed by consensus alignment e.g. by the methods described by Feng and Doolittle, Meth. Enzymol., Vol. 266, pp. 368-382 (1996); Feng and Doolittle, J. Mol. Evol., Vol. 25, pp. 351-360 (1987); and Taylor,. Meth. Enzymol., Vol. 266, pp. 343-367 (1996).

This leads to identification of epitope patterns, which can assist the comparison of the information obtained from the antibody binding peptide sequences to the 3-dimensional structure of the acceptor protein in order to identify epitope sequences at the surface of the acceptor protein.

Epitope Patterns

Given a number of antibody binding peptide sequences and possibly the corresponding epitope patterns, one need the 3-dimensional structure coordinates of an acceptor protein to find the epitope sequences on its surface.

These coordinates can be found in databases (NCBI: http://www.ncbi.nlm.nih.gov/), determined experimentally using conventional methods (Ducruix and Giegé: Crystallization of Nucleic Acids and Proteins, IRL PRess, Oxford, 1992, ISBN 0-19-963245-6), or they can be deduced from the coordinates of a homologous protein. Typical actions required for the construction of a model structure are: alignment of homologous sequences for which 3-dimensional structures exist, definition of Structurally Conserved Regions (SCRs), assignment of coordinates to SCRs, search for structural fragments/loops in structure databases to replace Variable Regions, assignment of coordinates to these regions, and structural refinement by energy minimization. Regions containing large inserts (>3 residues) relative to the known 3-dimensional structures are known to be quite difficult to model, and structural predictions must be considered with care.

One can match each amino acid residue of the antibody binding peptide to an identical or homologous amino acid on the 3-D surface of the acceptor protein, such that amino acids that are adjacent in the primary sequence are close on the surface of the acceptor protein, with is close being <10 Angstroms, particularly <5 Anstroms, more particularly <3 Angstroms between any two atoms of the two amino acids.

Alternatively, one can define a geometric body (e.g. an ellipsoid, a sphere, or a box) of a size that matches a possible binding interface between antibody and antigen and look for a positioning of this body where it will contain most of or all the anchor amino acids.

Also, one can use the epitope patterns to facilitate identification of epitope sequences. This can be done, by first matching the anchor amino acids on the 3-D structure and subsequently looking for other elements of the antibody binding peptide sequences, which provide additional matches. If there are many residues to be matched, it is only necessary that a suitable number can be found on the 3-D structure. For example if an epitope pattern comprises 4, 5, 6, or 7 amino acids, it is only necessary that 3 matches surface elements of the acceptor protein.

In all cases, it is desirable that amino acids of the epitope sequence are surface exposed (see Example 1).

How to Use the Epitope Information.

When applied on structurally and immunologically related immunogens, the information about epitope patterns and sequences, which can been derived by the above methods, can be utilized to assist in the selection of structural epitopes that are specific for the immunogen of interest.

After having identified the structural epitopes that will react with specific antibodies from a sample, which e.g. can be obtained from a human or an animal, these structural epitopes in the form of peptide sequences can be applied in a kit for testing binding of specific antibodies, particularly IgE antibodies, from the human or an animal, to the peptide sequences. This way it will be possible to predict binding of specific antibodies in a human or animal to structural epitopes comprised on potential immunogens.

Hence, in one embodiment the antigenic peptide to be employed in the kit of the invention is obtained by

(1) screening a random peptide library with antibodies raised against an immunogen of interest,

(2) determining the amino acid sequence of peptides binding to an antibody or the DNA sequences encoding the peptides,

(3) using the peptides or DNA sequences to identify at least one structural epitope pattern on the immunogen, and

(4) producing antigenic peptides corresponding to structural epitopes on the immunogen.

In a particular embodiment an antigenic peptide representing a structural epitope is a combination of one part of one antibody binding peptide combined with one or more parts from one or more different antibody binding peptides.

In a further embodiment the specificity or the affinity of antigenic peptides corresponding to structural epitopes on the immunogen may be increased by adding, deleting or mutating one or more amino acids in the sequence of the antigenic peptides or a combination thereof. Addition, deletion and mutation of amino acids in a sequence is known to the skilled person and may be achieved by conventional biochemical and/or genetic engineering methods.

Once the sequence of a suitable antigenic peptide representing a structural epitope has been identified, the peptides may be produced in any convenient way, e.g. by artificially synthesizing the peptides or expressing nucleic acid sequences encoding the peptides in a host.

Diagnostic Kit

Today, a patient suffering from an imunnogenic disease, such as allergy, may be subjected to allergy vaccine therapy using imunnogens selected on the basis of testing the specificity of the patient's serum IgE against a bank of immunogen extracts (or similar specificity tests of the patient's sensibilization such as skin prick test.

One could improve the quality of characterization by using antibody binding peptides corresponding to various epitope sequences on the protein imunnogens of interest. This would require a kit comprising reagents for such specificity characterization, e.g. the antibody binding peptides of desired specificity. It is particularly useful to use antibody binding sequences in the kit, which correspond to defined epitope sequences known to be specific for the immunogen under investigation (i.e. not identified on other immunogens and/or not cross-reacting with sera raised against other allergens). This kit would be useful to specifying which immunogenic decease, such as allergy, the patient is suffering from. This kit will lead to a more specific answer than those kits used today, and hence to a better selection of immunogen vaccine therapy for the individual patient.

In an extension of this approach, one could also characterize the patient's serum by identifying the corresponding antibody binding peptides among a random display library using the aforementioned methods. This again may lead to optimisation of the epitope information, and thus to a better diagnosis.

Further, one could use the individual antibody binding sequences as (immunogen) vaccines leading to more specific (immunogen) vaccines. These antibody binding sequences could be administered in an isolated form or fused to a membrane protein of the phage display system, or to another carrier protein, which may have beneficial effect for the immunoprotective effect of the antibody binding peptide (Dalum et al., Nature Biotechnology, Vol. 17, pp. 666-669 (1999)).

In a first aspect the present invention relates to a kit for predicting binding of a specific antibody to at least one potential immunogen, comprising

a) at least one antigenic peptide sequence comprising less than 26 amino acids wherein said antigenic peptide sequence corresponds to a structural epitope comprised in the at least one potential immunogen and the antigenic peptide sequence is capable of binding at least one antibody specific for the structural epitope comprised in the said potential immunogen, and

b) solid support suitable for immobilizing the at least one antigenic peptide sequence.

The kit of the invention would also be useful for other screening purposes where it is desirable to test for antibody binding to peptide sequences, e.g. for the development of epitope variants as mentioned previously.

Suitable solid support could in the present invention be any chemical support, including micro titer plates, beads, capillary tubing or membranes. Each of these supports could be activated, supporting covalent, ionic or hydrophobic binding, chelation or affinity binding, or inactivated, promoting ionic or hydrophobic binding.

Immobillisation could take place by attachment through covalent binding, ionic or hydrophobic binding, chelation, affinity binding, or through van der Waal bonds.

In the present invention, a solid support could also be biological in nature, such as phages, bacteria, red blood cells or any related system allowing display of heterologous proteins or peptides.

The above desribed kit can also be used for screening different antigenic peptide sequences corresponding to structural epitopes at the same time.

Given a number of proteins for which diagnosis optimally has to be performed simultaneously, a kit can be produced containing for each of these proteins a specific peptide corresponding to a structural epitope sequence comprised in the protein and immobilized on a solid support. As an example, 3 specific peptides are immobilized on beads, each peptide having its specific colored bead. In an agglutination format were specific antisera is mixed with this mixture of peptide coated beads, the color of the agglutinate will identify the specificity of the antibodies present in the patients serum.

In another embodiment the diagnostic kit comprises ten different antigenic peptide sequences and in a further embodiment the diagnostic kit comprises at least 100 different antigenic peptide sequences.

The kit above can also be used in a high throughput screening method for screening many samples, obtained e.g. from humans or animals, at the same time and thereby predicting which humans or animals will display an immunogenic response towards particular immunogens. Any practical combination of the number of antigenic peptide sequences and the number of humans or animals would be possible.

A second aspect of the invention therefore relates to a high throughput screening method for testing the presence of antibodies specific for a structural epitope comprised in at least one potential immunogen of interest, comprising

a) providing at least one antigenic peptide sequence comprising less than 26 amino acids wherein said antigenic peptide sequence(s) correspond(s) to one or more structural epitopes comprised in the at least one potential immunogen, and wherein the antigenic peptide sequence(s) is/are capable of binding at least one antibody specific for the structural epitope comprised in the said at least one potential immunogen,

b) immobilizing the at least one antigenic peptide sequences to a suitable solid support,

c) adding the specific antibodies from the human or animal, and

d) detecting binding of specific antibodies to any of the one or more antigenic peptide sequences.

In one embodiment antibodies from at least ten samples are screened and in another embodiment antibodies from at least 100 samples are screened.

Different Assay Formats are Compatible with a High Throughput Technology.

One such format is the ELISA format in for example 96, 384 or 1536 well plates. Another format is the agglutination format, where the relevant peptides are immobilized on (colored) beads or are presented by displaying organism, such as phages or bacteria. A third format is the blotting format, which uses membranes, such as nitrocellulose or polyvinyl-based membranes, as support. This format includes for example dot blot assays, and line immunoassays. A fourth assay format is the dipstick or pin based assays, were the peptide is immobilized on for example polystyrene or polyethylene pins.

If required, the solid supports can be activated chemically or biochemically in order to optimize binding of the target peptide to the support. This optimation might involve introduction of groups promoting covalent linkage, chelation, affinity binding, ionic or hydrophobic binding.

A chemical activation might for example lead to reactive NH₂ groups, or reactive Ni²⁺ complexes. Biochemical activation might include coating with avidin or streptavidin for cathing biotin-peptide complexes, short fatty acids for binding hydrophobic peptides, antibodies for binding biotin-labelled peptides.

A third aspect of the present invention relates to a use of the kit according to the invention for predicting binding of specific antibodies in a sample, e.g. obtained from a human or animal, to at least one potential immunogen, wherein binding to at least one antigenic peptide sequence corresponding to a structural epitope is tested. Particularly at least ten antigenic peptide sequences are tested, and in a further particular embodiment at least 100 antigenic peptide sequences are tested.

A fourth aspect of the present invention relates to a use of the high throughput screening method for screening antibodies from at least one sample, e.g. obtained from at least one human or animal, particularly from at least ten humans or animals, and in a further particular embodiment from at least 100 humans or animals.

Conformational/structural epitopes are as discussed previously composed of amino acid residues that are not all contained on the same contiguous amino acid sequence, but are brought into the right position to one another by folding of the protein. It is therefore possible for the distance between amino acids comprised on a structural epitope, but located on separated parts on the primary structure, to vary due to the dynamic nature of the conformation of the folded protein.

In the context of the present invention a structural epitope, comprised in the potential immunogen, comprises at least a first contiguous linear amino acid sequence consisting of at least one amino acid and a second contiguous linear amino acid sequence consisting of at least one amino acid, and wherein a distance between any two amino acids comprised in the structural epitope, which amino acids are not part of the same contiguous linear amino acid sequence, and which two amino acids are most proximal to each other, does not exceed 5 Angstroms.

In one embodiment the said distance should not exceed 3 Angstroms.

One way of measuring distances between amino acids on primary structures as well as 3D-structures of proteins uses Swissprot-PDBViewer (known by the skilled person in the art), which can be downloaded, free of charge, from www.expasy.com.

In one embodiment the first contiguous linear sequence and the second contiguous linear sequence are part of the same primary sequence of the immunogen. In case the first and second part of the epitope are both part of the same rimary sequence the first contiguous linear sequence and the second contiguous linear sequence are interrupted by at least one amino acid, particularly at least one amino acid which is located more than 10 Angstroms away from at least one amino acid of the first or second contiguous linear sequence. In a partcular embodiment the first contiguous linear sequence and the second contiguous linear sequence are interrupted by at least 10 amino acids.

In another embodiment the immunogen contains two or more subunits of primary sequences and the first contiguous linear sequence and the second contiguous linear sequence comprised in the epitope are part of two or more different primary sequences of the immunogen.

The epitope may contain more than two separated parts, such as three or four separated parts. However, in one embodiment the first contiguous linear sequence and the second contiguous linear sequence constitutes the structural epitope.

Cross-reactivity

In order to get a more specific and precise diagnosis, the antigenic peptide sequence, representing a structural epitopes on an immunogen, which is selected for the kit should display a minimal, e.g little or no cross-reactivity between the antibodies raised against an immunogen of interest and antibodies raised against any other ‘commercial’ and ‘environmental’ immunogen. When cross-reactivity is observed typically an antibody that will bind to one epitope (or antigenic peptide sequence representing the epitope) will also be able to bind to other epitopes, e.g. on other immunogens. Cross-reactivity is a common problem when using linear epitopes or antigenic peptide sequences representing a linear epitope in diagnostics. However when using antigenic peptide sequences representing a structural epitope in diagnostics, cross-reactivity is minimized.

In one embodiment the kit of the invention employs at least one antigenic peptide sequence, which corresponds to a structural epitope on at least one potential immunogen, wherein the at least one specific antibody, when present in excess with respect to the potential immunogen, will not bind to another antigen unless this antigen is present at a concentration which is 1000 fold higher than the potential immunogen.

In a further embodiment the kit of the invention employs at least one antigenic peptide sequence, which corresponds to a structural epitope on at least one potential immunogen, wherein the at least one antigenic peptide sequence has at least a 10 fold stronger affinity per microgram antigenic peptide towards at least one specific antibody in full blood or serum from an animal or human immunized with the full immunogen, than towards a non-specific antibody provided that the concentration of the specific antibody and the non-specific antibody is the same.

In a further embodiment the serum may be purified so as to mainly or completely contain antibodies of a selected class. Hence, the kit of the invention employs at least one antigenic peptide sequence, which corresponds to a structural epitope on at least one potential immunogen, wherein the antigenic peptide sequence has at least a 10 fold stronger affinity per microgram antigenic peptide towards at least one specific antibody in purified serum from an animal or human immunized with the full immunogen than towards a non-specific antibody provided that the concentration of the specific antibody and the non-specific antibody is the same, and wherein at least 50% of the specific antibodies present in the purified serum belongs to the same class of antibodies.

Particular classes of antibodies includes IgE, IgG, IgA, IgM or IgD. Further, the serum may be purified so that at least 75% of the antibodies in the purified serum, such as at least 90%, e.g. at least 98%, particularly at least 99% or even 100% belongs to the same class.

In a further embodiment the serum may be purified so as to mainly or completely contain antibodies which will bind to the employed antigenic peptides. Hense in this embodiment the kit of the invention employs at least one antigenic peptide sequence, which corresponds to a structural epitope on at least one potential immunogen, wherein the at least one antigenic peptide sequence has at least a 10 fold stronger affinity per microgram antigenic peptide towards at least one specific antibody in purified serum from an animal or human immunized with the full immunogen, than towards a non-specific antibody provided that the concentration of the specific antibody and the non-specific antibody is the same, and wherein at least 90% of the specific antibodies present in the purified serum binds to the at least one antigenic peptide sequence. Further, the serum may be purified so that at least 95% of the antibodies in the purified serum binds to the antigenic peptide sequence, in particular at least 98%, at least 99% or even 100%.

In the previous four embodiments the affinity of the antigenic peptide sequence may in particular be at least 20 fold stronger, more particularly at least 50 fold stronger affinity, more particularly at least 100 fold stronger. Further in these embodiments the at least one specific antibody towards which the antigenic peptide sequence has affinity is in particular a collection of 1-10 different antibodies, more particularly 1-5 different antibodies, more particularly 1-3 different antibodies. In a still further embodiment the at least one specific antibody is one specific antibody.

Cross-reactivities between food allergens of different origin are well-known (Akkerdaas et al, Allergy 50, pp 215-220, 1995). Similarly, cross-reactivities between other environmental allergens (like pollen, dust mites etc.) and commercial allergens (like enzyme proteins) have been established in the literature (J. All. Clin. Immunol., Vol. 102, pp. 679-686 (1998) and by the present inventors. The molecular reason for this cross-reactivity can be explored using epitope mapping.

The general principle of the present invention, whereby random peptide libraries are screened for any peptides capable of binding to specific antibodies, and these isolated random peptides subsequently are fitted by epitope mapping to 3D-models of known proteins thereby identifying first epitope patterns and second structural epitope sequences on the 3D-struture of the protein, and finally using a antigenic peptide sequence corresponding to the identified structural epitope for predicting binding of specific antibodies in a human or animal to a potential immunogen, can be applied for any kind of immunogen or immunogenic protein. Using other immunogens than those specifically mentioned will be obvious for the skilled person and is to be considered within the scope of the present invention.

In one embodiment the immunogen is an antigen, and particularly an allergen.

Immunogenic Protein or Immunogen

The “immunogenic protein” or “immunogen” can in principle be any protein molecule of biological origin, non-limiting examples of which are peptides, polypeptides, proteins, enzymes, post-translationally modified polypeptides such as lipopeptides or glycosylated peptides, anti-s microbial peptides or molecules, toxins, marker proteins of bacterial, viral or mammalian origin which indicate a specific disease, such as e.g. cancer or a specific infection, and proteins having pharmaceutical properties etc.

Accordingly in one embodiment, the “immunogen” is chosen from the group consisting of polypeptides, small peptides, lipopeptides, antimicrobials, toxins, marker proteins, pharmaceutical polypeptides, enzymes, industrial proteins and environmental allergens.

Particularly, the allergen is an enzymes or an environmental allergen or a pharmaceutical peptide.

The term “pharmaceutical polypeptides” is defined as polypeptides, including peptides, such as peptide hormones, proteins and/or enzymes, being physiologically active when introduced is into the circulatory system of the body of humans and/or animals.

Pharmaceutical polypeptides are potentially immunogenic as they are introduced into the circulatory system.

Examples of “pharmaceutical polypeptides” contemplated according to the invention include insulin, ACTH, glucagon, somatostatin, somatotropin, thymosin, parathyroid hormone, pigmentary hormones, somatomedin, erythropoietin, luteinizing hormone, chorionic gonadotropin, hypothalmic releasing factors, antidiuretic hormones, thyroid stimulating hormone, relaxin, interferon, thrombopoietin (TPO) and prolactin.

However, the proteins are particularly to be used in industry, housekeeping and/or medicine, such as proteins used in personal care products (for example shampoo; soap; skin, hand and face lotions; skin, hand and face cremes; hair dyes; toothpaste), food (for example in the baking industry), detergents and pharmaceuticals.

Antimicrobial Peptides

The antimicrobial peptide (AMP) may be, e.g., a membrane-active antimicrobial peptide, or an antimicrobial peptide affecting/interacting with intracellular targets, e.g. binding to cell DNA. The AMP is generally a relatively short peptide, consisting of less than 100 amino acid residues, typically 20-80 residues. The antimicrobial peptide has bactericidal and/or fungicidal effect, and it may also have antiviral or antitumour effects. It generally has low cytotoxicity against normal mammalian cells.

The antimicrobial peptide is generally highly cationic and hydrophobic. It typically contains several arginine and lysine residues, and it may not contain a single glutamate or asparatate. It usually contains a large proportion of hydrophobic residues. The peptide generally has an amphiphilic structure, with one surface being highly positive and the other hydrophobic.

The antimicrobial peptide may act on cell membranes of target microorganisms, e.g. through nonspecific binding to the membrane, usually in a membrane-parallel orientation, interacting only with one face of the bilayer.

The antimicrobial peptide typically has a structure belonging to one of five major classes: a helical, cystine-rich (defensin-like), beta-sheet, peptides with an unusual composition of regular amino acids, and peptides containing uncommon modified amino acids.

Enzymes

In one embodiment of the invention the protein is an enzyme or enzyme variant. It is to be understood that enzyme variants (produced, for example, by recombinant techniques) are included within the meaning of the term “enzyme”. Examples of such enzyme variants are disclosed, e.g., in EP 251,446 (Genencor), WO 91/00345 (Novo Nordisk), EP 525,610 (Solvay) and WO 94/02618 (Gist-Brocades NV).

Particularly the enzyme is selected from the group consisting of of glycosyl hydrolases, carbohydrases, peroxidases, proteases, lipolytic enzymes, phytases, polysaccharide lyases, oxidoreductases, transglutaminases and glucoseisomerases.

The enzyme classification employed in the present specification with claims is in accordance with Recommendations (1992) of the Nomenclature Committee of the International Union of Biochemistry and Molecular Biology, Academic Press, Inc., 1992.

Accordingly the types of enzymes which may appropriately be incorporated in granules of the invention include oxidoreductases (EC 1 . . . . ), transferases (EC 2 . . . ), hydrolases (EC 3 . . . ), lyases (EC 4 . . . ), isomerases (EC 5 . . . ) and ligases (EC 6 . . . ).

In particular oxidoreductases in the context of the invention are peroxidases (EC 1.11.1), laccases (EC 1.10.3.2) and glucose oxidases (EC 1.1.3.4)].

In particular transferases are transferases in any of the following sub-classes:

a) transferases transferring one-carbon groups (EC 2.1);

b) transferases transferring aldehyde or ketone residues (EC 2.2); acyltransferases (EC 2.3);

c) lycosyltransferases (EC 2.4);

d) transferases transferring alkyl or aryl groups, other that methyl groups (EC 2.5); and

e) transferases transferring nitrogeneous groups (EC 2.6).

A particular type of transferase in the context of the invention is a transglutaminase (protein-glutamine gamma-glutamyltransferase; EC 2.3.2.13). Further examples of suitable transglutaminases are described in WO 96/06931 (Novo Nordisk A/S).

In particular hydrolases in the context of the invention are: Carboxylic ester hydrolases (EC 3.1.1.-) such as lipases (EC 3.1.1.3-); phytases (EC 3.1.3.-), e.g. 3-phytases (EC 3.1.3.8) and 6-phytases (EC 3.1.3.26); glycosidases (EC 3.2, which fall within a group denoted herein as “carbohydrases”), such as alpha-amylases (EC 3.2.1.1); peptidases (EC 3.4, also known as proteases); and other carbonyl hydrolases.

In the present context, the term “carbohydrase” is used to denote not only enzymes capable of breaking down carbohydrate chains (e.g. starches or cellulose) of especially five-and six-membered ring structures (i.e. glycosidases, EC 3.2), but also enzymes capable of isomerizing carbohydrates, e.g. six-membered ring structures such as D-glucose to five-membered ring structures such as D-fructose. Carbohydrases of relevance include the following (EC numbers in parentheses): alpha-amylases (EC 3.2.1.1), beta-amylases (EC 3.2.1.2), glucan alpha-1,4-glucosidases (EC 3.2.1.3), beta-1,4-endoglucanase (cellulases, EC 3.2.1.4), endo-beta-1,3(4)-glucanases (EC 3.2.1.6), beta-1,4-endoxylanases (EC 3.2.1.8), dextranases (EC 3.2.1.11), chitinases (EC 3.2.1.14), polygalacturonases (EC 3.2.1.15), lysozymes (EC 3.2.1.17), β-glucosidases (EC 3.2.1.21), alpha-galactosidases (EC 3.2.1.22), beta-galactosidases (EC 3.2.1.23), amylo-1,6-glucosidases (EC 3.2.1.33), xylan beta-1,4-xylosidases (EC 3.2.1.37), glucan endo-beta-1,3-D-glucosidases (EC 3.2.1.39), dextrin endo-alpha-1,6-glucosidases (EC3.2.1.41), sucrose alpha-glucosidases (EC 3.2.1.48), glucan endo-alpha-1,3-glucosidases (EC 3.2.1.59), glucan beta-1,4-glucosidases (EC 3.2.1.74), glucan endo-beta-1,6-glucosidases (EC 3.2.1.75), arabinan endo-alpha-1,5-L-arabinosidases (EC 3.2.1.99), lactases (EC 3.2.1.108), chitosanases (EC 3.2.1.132) and xylose isomerases (EC 5.3.1.5).

In particular isomerases in the context of the invention are glycose isomerases

In particular lyases in the context of the invention are polysaccharide lyases.

Environmental Immunogens

The environmental immunogens that are of interest include allergens from pollen, dust mites, mammals, venoms, fungi, food items, and other plants.

Pollen, allergens include but are not limited to those of the order Fagales, Oleales, Pinales, Poales, Asterales, and Urticales; including those from Betula, Alnus, Corylus, Carpinus, Olea, Phleum pratense and Artemisia vulgaris, such as Aln g1, Cor a1, Car b1, Cry j1, Amb a1 and a2, Art v1, Par j1, Ole e1, Ave v1, and Bet v1 (WO 99/47680).

Mite allergens include but are not limited to those from Derm. farinae and Derm. pteronys., such as Der f1 and f2, and Der p1 and p2.

From mammals, relevant environmental allergens include but are not limited to those from cat, dog, and horse as well as from dandruff from the hair of those animals, such as Fel d1; Can f1; Equ c1 ; Equ c2; Equ c3.

Venum allergens include but are not limited to PLA2 from bee venom as well as Apis m1 and m2, Ves g1, g2 and g5, Ves v5 and te Pol and Sol allergens.

Fungal allergens include those from Alternaria alt. and Cladospo. herb. such as Alt a1 and Cla h1.

Food allergens include but are not limited to those from milk (lactoglobulin), egg (ovalbumin), peanuts, hazelnuts, wheat (alfa-amylase inhibitor).

Other plant allergens include latex (hevea brasiliensis).

The above described kit and high throughput screening method will be of great importance in order to more specifically identify the exact cause of an observed immunogenic response in a human or animal since the use of a antigenic peptide sequence corresponding to a structural epitope on an immunogen will give a much more specific answer than if a linear epitope was used. Also the identification of antigenic peptide sequences corresponding to structural epitopes on potential immunogens will facilitate the use of such antigenic peptide sequences in order to get more specific vaccines.

In a fifth aspect the present invention relates to a vaccine comprising at least one antigenic peptide sequence corresponding to a structural epitope comprised in at least one potential immunogen and said antigenic peptide sequence being capable of binding at least one antibody specific for a structural epitope comprised in a potential immunogen, and also in a sixth aspect to a method of preparing a vaccine comprising adding at least one antigenic peptide sequence corresponding to a structural epitope comprised in at least one potential immunogen and said antigenic peptide sequence being capable of binding at least one antibody specific for a structural epitope comprised in a potential immunogen to a liquid medium.

In a seventh aspect, the invention relates to the use of at least one antigenic peptide sequence, corresponding to a structural epitope comprised in at least one potential immunogen and said antigenic peptide sequence being capable of binding at least one antibody specific for a structural epitope comprised in a potential immunogen, for the preparation of a vaccine. Use of the vaccine of the invention for the treatment of a human or an animal also falls withing the scope of the present invention.

Materials and Methods

Materials

ELISA Reagents:

Horse Radish Peroxidase labelled pig anti-rabbit-Ig (Dako, DK, P217, dilution 1:1000).

Rat anti-mouse IgE (Serotec MCA419; dilution 1:100).

Mouse anti-rat IgE (Serotec MCA193; dilution 1:200).

Biotin-labelled mouse anti-rat IgG1 monoclonal antibody (Zymed 03-9140; dilution 1:1000)

Biotin-labelled rat anti-mouse IgG1 monoclonal antibody (Serotec MCA336B; dilution 1:2000).

Streptavidin-horse radish peroxidase (Kirkegard & Perry 14-30-00; dilution 1:1000).

Buffers and Solutions:

PBS (pH 7.2 (1 liter))

| | |
|---|---|
| NaCl | 8.00 g |
| KCl | 0.20 g |
| K₂HPO₄ | 1.04 g |
| KH₂PO₄ | 0.32 g |

Washing buffer PBS, 0.05% (v/v) Tween 20
Blocking buffer PBS, 2% (wt/v) Skim Milk powder
Dilution buffer PBS, 0.05% (v/v) Tween 20, 0.5% (wt/v) Skim Milk
powder
Citrate buffer 0.1 M, pH 5.0-5.2
Stop-solution (DMG-buffer)
Sodium Borate, borax (Sigma)
3,3-Dimethyl glutaric acid (Sigma)
Tween 20: Poly oxyethylene sorbitan mono laurate (Merck cat no.
822184)
PMSF (phenyl methyl sulfonyl flouride) from Sigma
Succinyl-Alanine-Alanine-Proline-Phenylalanine-paranitro-anilide (Suc-
AAPF-pNP) Sigma no. S-7388, Mw 624.6 g/mol.
mPEG (Fluka)

Coloring Substrate:

OPD: o-phenylene-diamine, (Kementec cat no. 4260)

Methods

Automatic Epitope Mapping

Implementation:

The implementation consists of 3 pieces of code:

1. The core program (see above), written in C (see Appendix A).

2. A “wrapping” cgi-script run by the web server, written in Python (see Appendix B).

3. A HTML page defining the input/submission form (see Appendix C).

The wrapper receives the input and calls the core program and several other utilities. Apart from the standard Unix utility programs (mv, rm, awk, etc.) the following must be installed:

A web server capable of running cgi-scripts, eg. Apache

Python 1.5 or later

Gnuplot 3.7 or later

DSSP, version July 1995

The Core Program:

Inputs

1. A Brookhaven PDB file with the structure of the protein

2. The output of DSSP called with the above PDB file.

3. Maximum distance between adjacent residues

4. Minimum solvent accessible surface area for each residue

5. Maximum epitope size (max distance between any two residues in epitope)

6. Maximum number of non-redundant epitopes to include (0=all)

7. The shortest acceptable epitope (as a fraction of the length of the epitope consensus sequence).

8. Epitope consensus sequence describing which residues are possible at the different positions. An example is shown below:

KR (Lys and Arg allowed)

AILV- (Ala, lIe, Leu, Val or missing residue allowed)

* (All residues allowed, but there must be a residue)

? (All or missing residue allowed)

DE (Asp or Glu allowed)

(*, ? or - in first or last position is allowed but obsolete. (- in first position is ignored.))

Examples of matching epitopes:

KAAKD (SEQ ID NO: 1), KLASD (SEQ ID NO: 2), KLYSD (SEQ ID NO: 3), KLY-D (SEQ ID NO: 4), R-M-D.

The Epitope Searching Algorithm:

The “core” of the program is the algorithm that scans the protein surface for the epitope patterns. The principle is that several “trees” are built, where each of their branches describes one epitope:

All residues in the protein are checked according to: a) Does the residue type match the first residue of the epitope consensus sequence. b) Is the surface accessibility greater than or equal to the given threshold. If both requirements are fulfilled, the protein residue is considered as one root in the epitope tree. Remark that there are usually many roots.

1. For each of the residues defined as roots, all residues within the the given threshold distance between adjacent residues (e.g. 7 Angstroms) are checked for the same as above: a) Does the residue type match the second residue of the epitope consensus sequence. b) Is the surface accessibility greater than or equal to the given threshold. If yes, the protein residue is considered as a “child” of the root. The spatial position of a residue is defined as the coordinates of its C-alpha atom.

2. The procedure from step 2 is repeated for the next residue in the epitope consensus sequence, where each of the “childs” found in step 2 are now “roots” of new childs. If a gap is defined in the epitope consensus sequence, a “missing” residue is allowed, and the coordinates of the root (also called “parent”) is used.

3. This procedure is repeated for all residues in the epitope consensus sequence.

4. In this way a number of trees (corresponding to the number of roots found in step 1) are found. Notice that the same protein residue can be present many places in the trees.

5. If no epitopes that matches the length of the epitope consensus sequence are found, the longest shorter epitopes that matches the first n residues of the epitope consensus sequence are used, where n is an integer smaller than the length of the epitope consensus sequence. If n is smaller than the length of the epitope consensus sequence multiplied by the fraction value defining the shortest acceptable epitope length, no epitopes are written to the output, and steps 7, 8 and 9 are skipped.

6. The epitopes are extracted from the trees by traversing down from each of the “childs” in the last level. The algorithm also finds epitopes which have the same protein residue present more than once. This is, of course, an artifact and such epitopes are discarded. Every epitope is then checked for its size, that is, the maximum distance between any two residues which are members of the epitope. If this exceeds the threshold, the epitope is discarded.

7. Redundant epitopes are removed. Epitopes containing one or more gaps are redundant if they are subsets of other epitopes without or with fewer gaps. For example: A82-gap-F45-G44-K43 is a subset of A82-L46-F45-G44-K43, and is therefore discarded.

8. For every epitope, the total solvent accessible surface area is calculated (by adding the contributions from each residue as found by the DSSP program). The epitopes are sorted according to this area in descending order. If a maximum number of n non-redundant epitopes has been specified, the n epitopes with largest solvent accessible surface area are selected.

9. The output consists of a list of the found epitopes, along with information of the epitope consensus sequence used and other internal parameters. A separate file containing the number of epitopes that each of the protein residues is a member of is also written.

The Wrapper:

Inputs

1. One PDB file, describing one structure, or one ZIP file, containing a number of PDB files, each describing one structure. The ZIP file must not contain subfolders.

2. An epitope consensus sequence or which part of the current epitope library to use (full library or IgE part or IgG part).

3. Maximum distance between adjacent residues

4. Minimum solvent accessible surface area for each residue

5. Maximum epitope size (max distance between any two residues in epitope)

6. Maximum number of non-redundant epitopes to include (0 =all)

7. Whether to use sequential numbering (1,2,3,4 . . . etc) or PDB-file numbering.

Description

The core program accepts only one structure and one epitope consensus sequence. It is usually desirable to use a library of epitope consensus sequences and sometimes several protein structures. The wrapper reads the user input and calls the utility programs and the core program the necessary number of times. The output is collected and presented on the web page returned to the user.

Depending on the type of input, the wrapper works in different modes:

Epitope consensus can be given directly or taken from a library

Input type can be a single PDB file or a collection of PDB file given as a ZIP-file.

Any of the four possible combinations are allowed.

The epitope library consists of a number of text files, each containing one epitope consensus sequence as specified above.

The layout of the wrapper is like this:

1. Check if the program is already in use from somewhere else (this is done by checking for a lock file when the wrapper starts. If it does not exist, it is created and removed again when the program is finished).

2. If the epitope consensus sequences are to be read from the library, make an internal list of the desired library entries.

3. If the input type is a ZIP file, unzip the file and create one new directory for each of the conatined PDB files. Move each PDB file to its corresponding directory.

4. Do a loop over the structures and/or epitope consensus sequences. For each structure/epitope consensus sequence pair, DSSP and the core program is called with the required parameters. If the input type is a ZIP file, the outputs are put in the appropriate directories.

5. If the epitope library is used, a sum file containing the total number of epitopes each residue is a member of. (Such a file is generated by the core program for each epitope consensus sequence—here a sum of these files is calculated). If input type is a ZIP file, a sum file is generated for each structure and put in the appropriate directory.

6. If the epitope library is used, a file containing the total number of epitopes found from each entry in the epitope library. If the input type is a PDB file, the file contains only one line (with a number of data corresponding to the library size). If the input type is a ZIP file, there is one line for each structure.

7. Depending on the combination of input type (ZIP or single PDB) and epitope consensus sequence source (typed-in or epitope library), different information is returned to the user:

Single PDB+typed in epitope: Graph of numbers of epitopes that each residue is a member of. List of found epitopes.

ZIP file+typed in epitope: Graphs (one for each structure) of numbers of epitopes that each residue is a member of. Lists (one for each structure) of found epitopes.

Single PDB+epitope library: Graph of numbers of epitopes that each residue is a member of (total for the complete library).

ZIP file+epitope library: Graphs (one for each structure) of numbers of epitopes that each residue is a member of (total for the complete library).

Data flow sheets for the four different are shown in the figure

8. For all modes except Single PDB+typed in epitope, a ZIP file containing all output files is created and returned to the user.

ELISA Procedure for Detecting Serum Levels of IgE and IgG:

Specific IgG and IgE levels were determined using the ELISA specific for human, mouse or rat IgG or IgE. Differences between data sets were analysed by using appropriate statistical methods. The assays were performed as known to the expert.

Activation of CovaLink Plates:

A fresh stock solution of cyanuric chloride in acetone (10 mg/ml) is diluted into PBS, while stirring, to a final concentration of 1 mg/ml and immediately aliquoted into CovaLink NH2 is plates (100 microliter per well) and incubated for 5 minutes at room temperature. After three washes with PBS, the plates are dryed at 50° C. for 30 minutes, sealed with sealing tape, and stored in plastic bags at room temperature for up to 3 weeks.

Protein Sequences and Alignments:

For purposes of the present invention, the degree of homology may be suitably determined by means of computer programs known in the art, such as GAP provided in the GCG program package (Program Manual for the Wisconsin Package, Version 8, August 1994, Genetics Computer Group, 575 Science Drive, Madison, Wis., USA 53711) (Needleman and Wunsch, Journal of Molecular Biology, Vol. 48, 443-45 (1970)).

Examples of alignments are described in WO 01/83559.

Structures

The structure of Savinase® can be found in Betzel et al., J.Mol. Biol., vol. 223, p. 427 (1992) (1svn.pdb).

Homology Modelling

As described earlier one needs the 3-dimentional structure coordinates of an acceptor protein to find the epitope sequences on its surface. These coordinates if not already in a database can be deduced from the coordinates of a homologous protein. Typical actions required for the construction of a model structure are: alignment of homologous sequences for which 3-dimensional structures exist, definition of Structurally Conserved Regions (SCRs), assignment of coordinates to SCRs, search for structural fragments/loops in structure databases to replace Variable Regions, assignment of coordinates to these regions, and structural refinement by energy minimization.

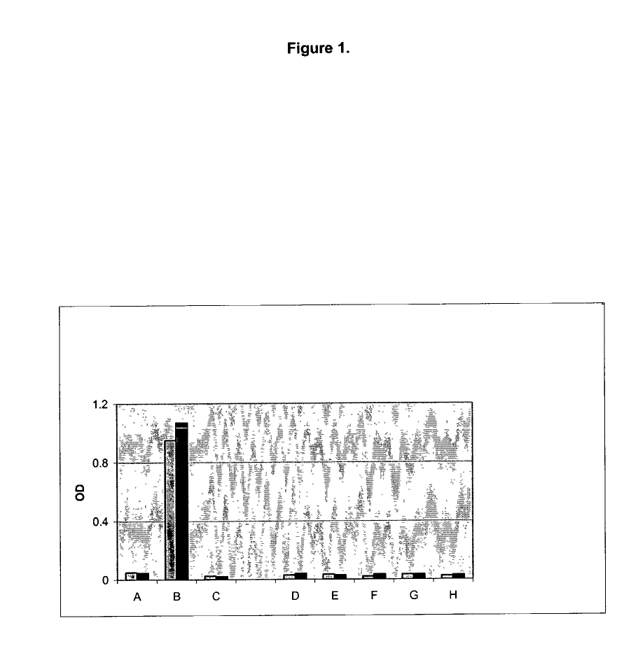

Examples of 3D-structural models are described in WO 01/83559, where three dimensional structural models of the subtilisins properase, relase, ProteaseC, ProteaseD, ProteaseE, and PROTEASE B were constructed based on three dimensional structure of Savinase® (Protein Data Bank entry 1SVN; Betzel, C., Klupsch, S., Papendorf, G., Hastrup, S., Branner, S., Wilson, K. S.: Crystal structure of the alkaline proteinase Savinase® from Bacillus lentus at 1.4 Å resolution, J. Mol. Biol., Vol. 223, pp. 427 (1992)) using the Modeller 5o (\overline{S}ali, A.; T. L. Blundell, “Definition of general topological equivalence in protein structures: A procedure involving comparison of properties and relationships through simulated annealing and dynamic programming,” J. Mol. Biol., Vol. 212, 403-428 (1990)) module of the Insight 2000 molecular modelling package (Biosym inc.). Default parameters were used with the alignments shown in FIG. 1A (WO 01/83559) as input, e.g. alignment between the columns labelled Savinase® and PROTEASE B served as input alignment in construction of a PROTEASE B structural model. The Modeller module by default output ten structural models, of these the model with lowest ‘modeller objective function’ score was chosen as representing PROTEASE B structure.

The amylase used in the examples of WO 01/83559 is the alpha-amylase of Bacillus halmapalus (WO 96/23873), which is called amylase SP722 (the wild-type). Its sequence is shown in SEQ ID NO 2 (WO 01/83559) and the corresponding protein structure was built from the BA2 structure, as described in WO 96/23874. The first four amino acids of the structural model are not defined, hence the sequence used for numeration of amino acid residues in the examples of this invention is four amino acids shorter than the one of the full length protein SP722.

Several variants of this amylase are available (WO 96/23873). One particularly useful variant has deleted two amino acid residues at D-G at positions 183 and 184 of SEQ ID NO 2 of WO 01/83559 (corresponding to residues 179 and 180 of the modelled structure). This variant is called JE-1 or Natalase.

Another amylase that is particularly useful is the amylase M560: This alkaline alpha-amylase may be derived from a strain of Bacillus sp. DSM 12649. The strain was deposited on Jan. 25, 1999 by the assignee under the terms of the Budapest Treaty on the International Recognition of the Deposit of Microorganisms for the Purposes of Patent Procedure at Deutshe Sammmlung von Microorganismen und Zellkulturen GmbH (DSMZ), Mascheroder Weg 1b, D-38124 Braunschweig DE.

### EXAMPLES

### Example 1

From a phage display library expressing random hexa-, nona- or dodeca peptides as part of their membrane proteins, specific phage clones were isolated capable of binding specific antibodies. The DNA sequence encoding the displayed peptide of one such clone was determined according to standard procedure. The amino acid sequence of the corresponding oligopeptide was deduced from the DNA sequence. This analysis revealed the peptide VQVYGDTSA (SEQ ID NO: 5) as a specific antibody binding peptide.

Epitope Pattern.

By sequence alignment using the “geometric body” approach as described earlier, the epitope pattern: Q206>Y214>D41> was localized on the 3D structure of Savinase®.

The identified epitope pattern, Q206>Y214>D41>, was then fitted with the 3D-structure of Savinase®.

Epitope Sequence.

The potential epitope sequence was identified by incorporation of the non-anchor amino acids identified by the phage display (settings: 100% homology, and >20 Å² accessibility for each amino acid). This identified the potential epitope sequence: Q206 V81 Y214 G80 D41 T208.

Detailed description of how to map epitopes and identify potential epitope sequences is also disclosed in WO 00/26230 and WO 01/83559 the content of which is hereby incorporated by reference.

### Example 2

Epitope mapping was also used to identify epitope patterns specific for Alcalase®, Savinas®, and Subtilisin Novo®. These proteases crossreact significantly in ELISA using specific rabbit antibody. The specific epitope patterns are shown in Table 1 below and epitope patterns, which are specific for each of these proteases, are underlined.

TABLE 1

| Alcalase ® | Savinase ® | Subtilisin Novo ® |
|---|---|---|
| | | Epi#04 |
| Epi#05 | Epi#05 | Epi#05 |
| | | Epi#06 |
| Epi#08 | | |
| Epi#09 | Epi#09 | Epi#09 |
| | Epi#10 | |
| Epi#12 | Epi#12 | |
| | | Epi#14 |
| Epi#17 | Epi#17 | Epi#17 |
| Epi#18 | Epi#18 | Epi#18 |
| Epi#19 | Epi#19 | Epi#19 |
| Epi#22 | | Epi#22 |
| Epi#23 | Epi#23 | |
| | | Epi#24 |
| Epi#25 | | |
| | | Epi#26 |
| EDi#27 | | |
| Epi#28 | Epi#28 | |
| | | Epi#29 |
| | | Epi#30 |
| | Epi#31 | Epi#31 |
| Epi#32 | | |
| Epi#33 | | Epi#33 |
| | Epi#34 | Epi#34 |
| | | Epi#35 |
| Epi#36 | | Epi#36 |
| Epi#37 | Epi#37 | |
| Epi#40 | Epi#40 | Epi#40 |
| | | Epi#41 |
| Epi#42 | | |
| Epi#44 | Epi#44 | Epi#44 |
| | | Epi#45 |
| | Epi#46 | |
| | Epi#47 | |
| | | Epi#48 |
| | Epi#53 | Epi#53 |

### Example 3

From the example above, epitope#10 appears to be specific only for Savinase®, and this epitope can be translated into the following structural epitope sequences on the 3D-structure of the protease:

R180 F183 S182 N179 A173 T218 A156

R180 F183 S182 N179 D175 H220 T218

3D imaging of the protease showed that the epitopes were localized on the surface of the 3D structure.

The following sequences were synthezised, and immobilized on biotin, through a linker molecule:

R R F A N D H T R (SEQ ID NO: 6) and

R R F S N A T R A (SEQ ID NO: 7)

Alternatively, these sequences were cloned into the P8 membrane protein of phage lambda.

The biotin-complex was immobilized in ELISA well plates, pre-coated with streptavidin. If phages were used, these were directly coated into the ELISA plate wells. ELISA was performed as described elsewhere, on sera from rabbits, rats, and mice raised against Alcalase®, Savinase®, and Subtilisin Novo® as well as a number of less relevant proteins.

In FIG. 1 the reactivity of the selected peptides in terms of antibody binding capacity is shown (ELISA assay).

The different proteins are marked by capital letters A through H. A=Alcalase®, B=Savinase®, C=Subtilisin Novo®, D=Carezyme® (cellulase), E=Laccase, F=Natalase® (amylase), G=SP722 (amylase), H=Lipolase® (lipase).

The light gray bar represents the sequence R R F A N D H T R (SEQ ID NO: 6), and the dark gray bar represents the sequence R R F S N A T R A (SEQ ID NO: 7).

Reactivity was observed with anti-Savinase® antibody only, demonstrating the specificity of both linear antigenic peptide sequences corresponding to the two structural epitope sequences: R R F A N D H T R (SEQ ID NO: 6) and R R F S N A T R A (SEQ ID NO: 7).

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

1. A kit for predicting binding of a specific antibody to at least one potential immunogen, comprising
a) at least one antigenic peptide sequence comprising less than 26 amino acids wherein said antigenic peptide sequence corresponds to a structural epitope comprised in the at least one potential immunogen and the antigenic peptide sequence is capable of binding at least one antibody specific for the structural epitope comprised in the said potential immunogen, and
b) solid support suitable for immobilizing the at least one antigenic peptide sequence.

2. The kit of claim 1, wherein the structural epitope, comprised in the potential immunogen, comprises a first contiguous linear amino acid sequence consisting of at least one amino acid and a second contiguous linear amino acid sequence consisting of at least one amino acid, and wherein a distance between any two amino acids comprised in the structural epitope, which amino acids are not part of the same contiguous linear amino acid sequence, and which two amino acids are most proximal to each other, does not exceed 5 Angstroms.

3. The kit of claim 2, wherein the distance does not exceed 3 Angstroms.

4. The kit of claim 2 or 3, wherein the first contiguous linear sequence and the second contiguous linear sequence are part of the same primary sequence of the immunogen.

5. The kit of claim 4, wherein the first contiguous linear sequence and the second contiguous linear sequence are interrupted by at least one amino acid.

6. The kit of claim 5, wherein said at least one amino acid is located more than 10 Angstroms away from at least one amino acid of the first or second contiguous linear sequence.

7. The kit of claim 5, wherein the first contiguous linear sequence and the second contiguous linear sequence are interrupted by at least 10 amino acids.

8. The kit of claim 2 or 3, wherein the first contiguous linear sequence and the second contiguous linear sequence are part of different primary sequences of the immunogen.

9. The kit of any of the preceding claims, wherein the first contiguous linear sequence and the second contiguous linear sequence constitutes the structural epitope.

10. The kit of any of the preceding claims, wherein the at least one specific antibody, when present in excess with respect to the potential immunogen, will not bind to another antigen unless this antigen is present at a concentration which is 1000 fold higher than the potential immunogen.

11. The kit of any of the preceding claims, wherein the at least one antigenic peptide sequence has at least a 10 fold stronger affinity per microgram antigenic peptide towards at least one specific antibody in full blood or serum from an animal or human immunized with the full immunogen, than towards a non-specific antibody provided that the concentration of the specific antibody and the non-specific antibody is the same.

12. The kit of any of the preceding claims, wherein the at least one antigenic peptide sequence has at least a 10 fold stronger affinity per microgram antigenic peptide towards at least one specific antibody in purified serum from an animal or human immunized with the full immunogen than towards a non-specific antibody provided that the concentration of the specific antibody and the non-specific antibody is the same, and wherein at least 50% of the specific antibodies present in the purified serum belongs to the same class of antibodies.

13. The kit of claim 12, wherein the class of antibodies is selected from the group of IgE, IgG, IgA, IgM and IgD.

14. The kit of any of the preceding claims, wherein the at least one antigenic peptide sequence has at least a 10 fold stronger affinity per microgram antigenic peptide towards at least one specific antibody in purified serum from an animal or human immunized with the full immunogen, than towards a non-specific antibody provided that the concentration of the specific antibody and the non-specific antibody is the same, and wherein at least 90% of the specific antibodies present in the purified serum binds to the at least one antigenic peptide sequence.

15. The kit of any of the preceding claims, wherein the at least one antigenic peptide is obtained by screening a random peptide library with antibodies raised against an immunogen of interest and determining the amino acid sequence of peptides binding to an antibody or the DNA sequence encoding the peptides and producing said peptides.

16. The kit of any of the preceding claims, wherein the at least one antigenic peptide is obtained by
a) screening a random peptide library with antibodies raised against an immunogen of interest,
b) determining the amino acid sequence of peptides binding to an antibody or the DNA sequences encoding the peptides,
c) using the peptides or DNA sequences to identify at least one structural epitope pattern on the immunogen and
d) producing antigenic peptides corresponding to structural epitopes on the immunogen.

17. The kit of claim 16, wherein the antigenic peptide is a combination of one part of one antibody binding peptide combined with one or more parts from one or more different antibody binding peptides.

18. The kit of claim 16, wherein specificity or the affinity of the antigenic peptides corresponding to structural epitopes on the immunogen is increased by adding, deleting or mutating one or more amino acids in the sequence of the antigenic peptides or a combination thereof.

19. The kit of any of claims 15-18, wherein said producing of peptides is achieved by artificially synthesizing the peptides or expressing nucleic acid sequences encoding the peptides in a host.

20. The kit of claim 15, wherein the random peptide library is a display package library.

21. The kit of claim 20, wherein the peptide display package library is a phage display library.

22. The kit of any of claims 15-21, wherein the peptides of the random peptide library or the peptide display package library are oligopeptides having from 5-25 amino acids.

23. The kit of claim 22, wherein the peptides of the said library are oligopeptides having from 8-12 amino acids.

24. The kit of any of the preceding claims, wherein the at least one antigenic peptide is identified by structural epitope mapping.

25. The kit of any of the preceding claims, wherein the potential immunogen is an allergen.

26. The kit of claim 25, wherein the specific antibody is IgE antibody.

27. The kit of claim 25, wherein the allergen is an enzyme or an environmental allergen or a pharmaceutical polypeptide.

28. The kit of any of claims 1-24, wherein the potential immunogen is a marker specific for a disease such as cancer.

29. The kit of any of claims 1-24, wherein the potential immunogen is a toxin.

30. The kit of any of claims 1-24, wherein the potential immunogen is a marker specific for a bacterial or a viral infection.

31. The kit of claim 27, wherein the enzyme is selected from the group consisting of glycosyl hydrolases, carbohydrases, peroxidases, proteases, lipolytic enzymes, phytases, polysaccharide lyases, oxidoreductases, transglutaminases and glucoseisomerases.

32. The kit of claim 27, wherein the environmental allergen is selected from the group consisting of pollen, dust, mite, mammal, venom, fungal, or food allergens or other plant allergens.

33. The kit of claim 27, wherein the pharmaceutical polypeptide is selected from the group comprising insulin, ACTH, glucagon, somatostatin, somatotropin, thymosin, parathyroid hormone, pigmentary hormones, somatomedin, erythropoietin, luteinizing hormone, chorionic gonadotropin, hypothalmic releasing factors, antidiuretic hormones, thyroid stimulating hormone, relaxin, interferon, thrombopoietin (TPO) and prolactin.

34. The kit of any of the preceding claims, comprising at least two different antigenic peptide sequences.

35. The kit of any of the preceding claims, comprising at least 10 different antigenic peptide sequences.

36. The diagnostic kit of any of the preceding claims, comprising at least 100 different antigenic peptide sequences.

37. A high throughput screening method for testing the presence of antibodies specific for a structural epitope comprised in at least one potential immunogen of interest, comprising testing specific antibodies in the kit of any of claims 1-36.

38. A use of the high throughput screening method of claim 37, for screening antibodies from at least one sample.

39. A use of the high throughput screening method of claim 37, for screening antibodies from at least ten samples.

40. A use of the high throughput screening method of claim 37, for screening antibodies from at least 100 samples.

41. A use of the kit of any of claims 1-36, for predicting binding of specific antibodies in a sample to at least one potential immunogen, wherein binding to at least one antigenic peptide sequence is tested.

42. A use of the kit of any of claims 1-36, for predicting binding of specific antibodies in a sample to at least one potential immunogen, wherein binding to at least ten antigenic peptide sequences are tested.

43. A use of the kit of any of claims 1-36, for predicting binding of a specific antibody to at least one potential immunogen, wherein binding to at least 100 antigenic peptide sequences are tested.

44. A vaccine comprising at least one antigenic peptide sequence corresponding to a structural epitope comprised in at least one potential immunogen and said antigenic peptide sequence being capable of binding at least one antibody specific for the structural epitope comprised in the potential immunogen.

45. A method for the preparation of a vaccine comprising adding to a liquid medium at least one antigenic peptide sequence, corresponding to a structural epitope comprised in at least one potential immunogen and said antigenic peptide sequence being capable of binding at least one antibody specific for the structural epitope comprised in the potential immunogen.

46. A use of at least one antigenic peptide sequence, corresponding to a structural epitope comprised in at least one potential immunogen and said antigenic peptide sequence being capable of binding at least one antibody specific for the structural epitope comprised in the potential immunogen, for the preparation of a vaccine

47. A use of the vaccine of claim 44, for the treatment of a human or an animal.
